(19) United States
(12) Patent Application Publication
(10) Pub. No.: US 2007/0198090 A1
(43) Pub. Date: Aug. 23, 2007

(54) Use of Carbon Nanotubes in the Manufacture of Orthopedic Implants

(76) Inventor: M. S. Abdou, San Diego, CA (US)

Correspondence Address: FISH & RICHARDSON, PC, P.O. BOX 1022, MINNEAPOLIS, MN, 55440-1022, US

(21) Appl. No.: 11/671,391

(22) Filed: Feb. 5, 2007

Related U.S. Application Data
(60) Provisional application No. 60/765,440, filed on Feb. 3, 2006.

(51) Int. Cl. A61F 2/44; A61B 17/70; A61B 17/80

(52) U.S. Cl. 623/17.11; 606/76; 623/23.39; 606/61; 606/69

(57) ABSTRACT

A carbon nanotube is used to at least partially manufacture an orthopedic implant of improved strength and durability. The nanotubes are generally free of structural imperfections, possess high stiffness, high strength, low density, small size, excellent electrical properties as well as variable magnetic characteristics.

### REFERENCE TO PRIORITY DOCUMENT

This application claims priority of U.S. Provisional Patent Application Ser. No. 60/765,440, filed Feb. 3, 2006. Priority of the aforementioned filing date is hereby claimed and the disclosure of the Provisional Patent Application is hereby incorporated by reference in its entirety.

### BACKGROUND

The present disclosure is related to materials used in the manufacture of medical implants in general and orthopedic devices in particular.

The implantation of medical devices is common in current medical practice. These implants encompass a wide range of distinct devices that collectively provide varied biologic functions. Regardless of the intended function, the implant is desirably biocompatible and desirably produces minimal biological toxicity with prolonged use. These devices have been manufactured from metals, metallic alloys, ceramics, plastics, biologic materials, composite materials and the like. With growing experience, the performance characteristics of each of these substances within living organisms has been defined in detail.

The intended function of the implant will determine the biologic environment of implantation and the demands placed on the materials of manufacture. Orthopedic implants, for example, are commonly used to support and brace the bony elements, reinforce or replace the ligamentous structures, and function as bearings surface in the repair or replacement of joints. In these applications, the implants desirably possess a high load-bearing capacity and low wear characteristics. In addition, many of these implants are placed adjacent to fractured bone and desirably permit ongoing and unhindered x-ray evaluation of the healing bone. Also, as disclosed in U.S. Patent Publication Number 2002/0049394, some orthopedic implants have electronic subunits that are designed to record particular measurements that are integral to the therapeutic plan.

Because of the load-bearing requirements, metals and metallic alloys have been the most common materials used in the manufacture of orthopedic implants. While these materials provide significant strength per unit volume, they are also disadvantageously radio-opaque and obscure the underlying bone during post-operative x-ray evaluation. Because of this significant disadvantage, radiolucent thermoplastics, such as Polyetheretherketones (PEEK), have been used in some orthopedic implants. Unfortunately, the PEEK implant must be significantly thicker than the metallic implant in order to achieve comparable strength. Due to anatomical constraints, use of the thicker PEEK implants may be problematic or completely impossible in many applications.

### SUMMARY

In view of the foregoing, there is a need in the art for the development of orthopedic implants of composite materials that have greater strength per unit volume than is currently achievable. The material desirably permits unobstructed x-ray evaluation of the surrounding bone when implanted in a patient.

In an embodiment, carbon nanotubes are added to materials of manufacture in order to produce an orthopedic implant of improved strength and durability. These nanotubes are generally free of structural imperfections, possess high stiffness, high strength, low density, small size, excellent electrical properties as well as variable magnetic characteristics. While carbon nanotubes have been most extensively studied, other nanotube-like structures have been made from other elements such as, for example, boron (so called “boron whiskers”). Such nano-tube like structures can also be used to materials of manufacture.

In one aspect, there is disclosed a device for the stabilization of adjacent bony and ligamentous elements, comprising a body adapted to be at least partially situated between adjacent bony elements of a patient, the body defining at least one cavity adapted to contain a bone graft for bone fusion, wherein the body contains a superstructure adapted to support the load between the bony elements and wherein the body is at least partially manufactured from a nanotube.

In another aspect, there is disclosed a device for the stabilization of adjacent bony and ligamentous elements, comprising a body adapted to at least partially rest against a sidewall of a bony elements and affix to a pair of bony elements in a cantilevered fashion wherein the body is at least partially manufactured from a nanotube.

In another aspect, there is disclosed a device for the stabilization of a adjacent bony and ligamentous elements, comprising a body adapted to be at least partially situated between two bones wherein the body forms a bearing surface between the two bones wherein the body is at least partially manufactured from a nanotube.

In another aspect, there is disclosed a device for the stabilization of a adjacent bony and ligamentous elements, comprising a malleable tether used for stabilization of adjacent bones, wherein the tether provides the function of a natural ligament and wherein the tether is at least partially manufactured of a nanotube.

Other features and advantages will be apparent from the following description of various methods and will illustrate, by way of example, the principles of the disclosed invention.

### BRIEF DESCRIPTION OF THE DRAWINGS

FIG. 1A shows a perspective view of a first embodiment of a cage implant device that is at least partially manufactured of nanotubes.

FIG. 1B shows the cage implant device in use within a disc space between two vertebral bodies.

FIG. 2 shows the C60 spherical molecule.

FIG. 3A shows a single walled carbon nanotube (SWCNT) with a low aspect ratio.

FIG. 3B shows a close-up of the nanotube of FIG. 3A.

FIG. 4 illustrates a Multi-walled carbon nanotubes (MWCNT).

FIG. 5 shows an exemplary bone-support device comprised of a plate that uses bone screws or similar fasteners to attach onto the anterior aspect of the spine in cantilever fashion.

FIG. 6 shows a mobile prosthesis that is situated between two skeletal segments and used to at least partially replace a joint structure.

FIGS. 7A and 7B show two additional nanotube shapes.

FIGS. 8A and 8B illustrate side and top views of gear-like nano-tube devices.

FIG. 9 illustrates a nano-scale bearing surface.

FIGS. 10A and 10B show a nano-tube based gear assembly that contains complimentary benzyne teeth.

### DETAILED DESCRIPTION

Disclosed are methods and devices that are adapted to assist in the fusion of adjacent bones of a skeletal system. The methods and devices are described herein in the context of use in the spine, although the disclosed methods and devices are suitable for use in any skeletal region.

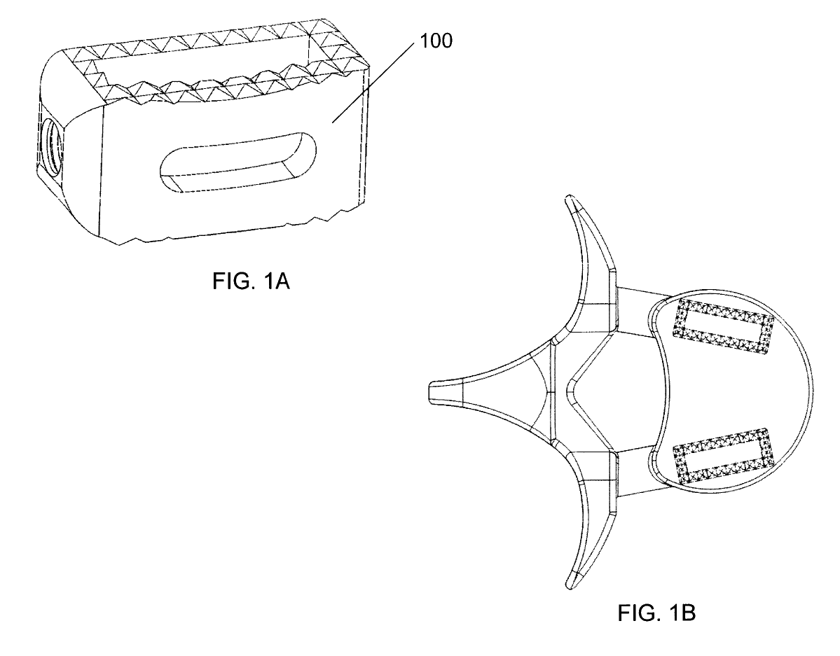

The devices can be used in various locations of a skeletal system. For example, in one embodiment the device is a cage configured to contain bone graft that fuses to one or more adjacent bones of a skeletal system in which the bones are located. FIG. 1A shows a perspective view of a first embodiment of a cage implant 100. FIG. 1B shows the cage implant 100 device in use within a disc space between two vertebral bodies. The cage implant 100 is sized and shaped to be implanted between an upper vertebra and a lower vertebra of a spine.

The cage implant 100 includes a main body configured to contain bone graft. The cage implant 100 is configured to be implanted between a pair of bones, such as vertebrae, so as to provide structural support and encourage fusion between the bones and bone graft contained within the cage implant 100. As mentioned, the cage implant 100 is described herein in an exemplary embodiment where the cage implant is positioned between two vertebrae, although it should be appreciated that the cage implant 100 can be used with other bones in a skeletal system.

The cage implant has a dual function in that it serves as a spacer between the bones while the hollow interior of the cage implant houses and contains the bone graft necessary for fusion. Since the long-term success of the operation is critically dependant on the formation of an adequate fusion mass, it is important that the interior chamber size and the amount of bone graft within it are maximized.

The device walls are desirably sufficiently strong so that they can support the extensive loads applied across the disc space but are also sufficiently thin in order to form an inner cavity of sufficient size for maximum bone graft volume. These constraints are most easily satisfied by manufacturing the device from metals or metallic alloys. Unfortunately, metals are radio-opaque and will not permit adequate visualization of the bone graft contained within the device. This is a significant disadvantage, since post-operative evaluation of the healing fusion mass requires periodic X-ray examination. More recently, skeletal implant devices have been manufactured from thermoplastic materials, such as polyetheretherketones (PEEK). Unfortunately, the thermoplastics materials are weaker than the metals and the device walls must be made significantly thicker in order to accommodate the inter-vertebral load. The large wall thickness of the PEEK devices leave little interior space for the bone graft and negate the very purpose of the fusion device. Carbon fiber reinforcement of the PEEK material has been used, but the device remains substantially weaker and the inner cavity remains substantially smaller than in those device manufactured from metals.

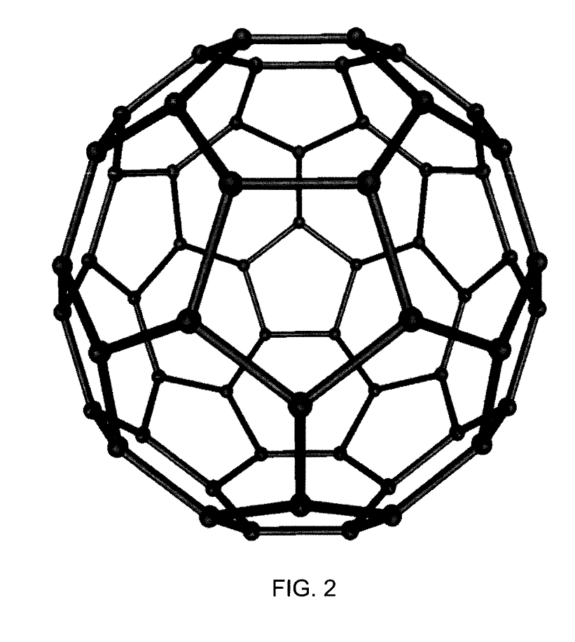

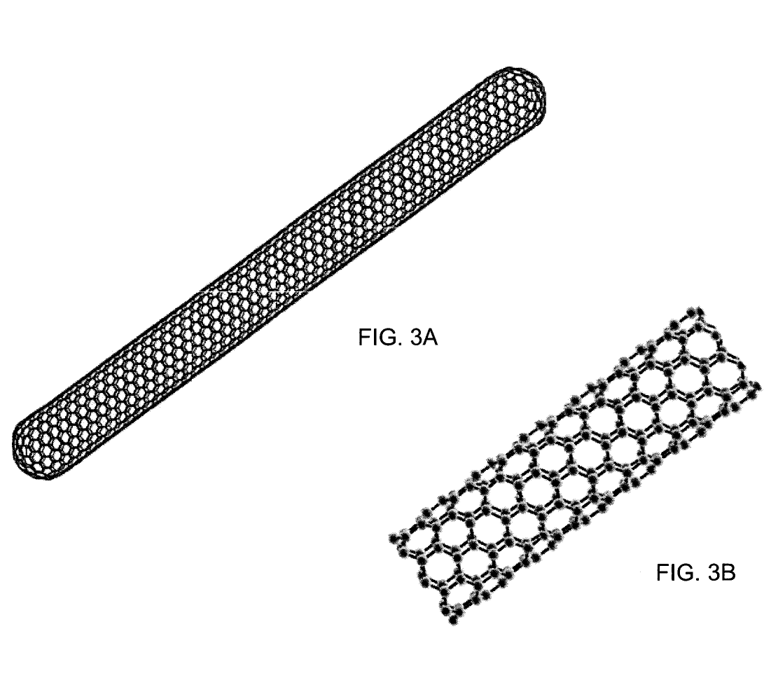

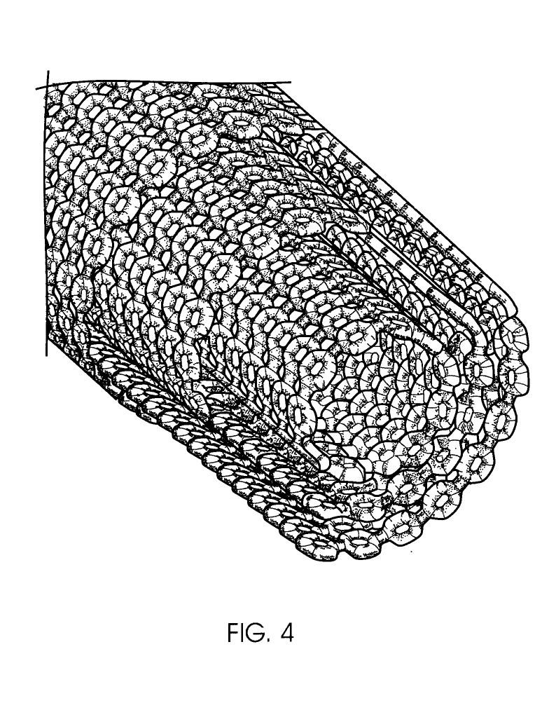

Composite materials that incorporate nanofibers in general, and carbon nanofibers in particular, significantly increase material strength and can be employed to effectively create a strong but thin device wall. Carbon nanotubes, an allotrope of carbon, are members of the Fullerene family. In general, the Fullerenes are molecules composed entirely of carbon and configured into seamless spherical or cylindrical shapes. The former include the extensively-studied C60 spherical molecule commonly known as the “buckyball” (FIG. 2) while the latter are collectively termed carbon nanotubes. Carbon nanotubes are further subdivided into single walled carbon nanotubes (SWCNT) and Multi-walled carbon nanotubes (MWCNT). FIG. 3A shows a SWCNT with a low aspect ratio while FIG. 3B shows a close-up of the nanotube. FIG. 4 illustrates a MWCNT. The SWCNT are comprised of a sheet of graphite of approximately 1 nanometer thickness (one atom thick) that are rolled into a cylindrical configuration with a semi-spherical cap at each end. The tubes commonly have a large aspect ratio so that the length of each tube is substantially larger than its diameter.

Since they're comprised of graphite sheets, each carbon atom within a carbon nanotube is covalently bonded to three other carbon atoms and contains a sp2 orbital hybridization. Unlike graphite, however, the sheets are not bound together by week forces but, instead, each sheet is rolled into a strong cylindrical configuration. Collectively, the sp2 orbital hybridization, which is stronger than the sp3 orbital hybridization of diamond, and the unique spatial configuration of the nanotubes impart novel properties upon them. These properties include phenomenal strength and stiffness, excellent electrical conductivity and an exceptional ability to return to the original configuration after deformation. Experimental and theoretical data have estimated Young's module at approximately 1 tera-pascel, compared with about 100 Mega-pascal for conventional carbon fiber and 1.2 giga-pascal for high-carbon steel. It is these intriguing properties that make carbon nanotubes particularly useful in the manufacture of orthopedic implants. A more thorough description of these molecules is detailed in the text “Carbon Nanotubes and Related Structures” by Peter J. F. Harris, and published by University Press, Cambridge, UK in 2003. The text is hereby incorporated by reference in its entirety.

Carbon nanotubes may be produced by a variety of techniques. In the most elementary process, these molecules can be produced in the uncontrolled environment of an ordinary flame and can be found in the deposited soot. However, this crude technique produces nanotubes with excessive imperfections and of varied sizes so that the procedure is of little value in commercial applications. Carbon nanotubes were first recognized after production by an arc-evaporation technique in 1991 in which graphite electrodes where held a short distance apart during electrical arcing. This technique remained a common method of production throughout the early 1990's.

Alternative techniques were soon developed and included the evaporation of graphite using an electron bean or laser ablation. The chemical vapor deposition technique and other electrochemical methods of synthesis have also been developed. These and other methods are well described in the above referenced text and in article from the journal “Composite science and technology” in the special issue “Nano-composites”, Volume 66, issue 9, 2006. The text is hereby incorporated by reference in its entirety. Further, a number of commercial ventures will now sell carbon nano-tube composites directly to interested parties. These companies include e-Spin Technologies, Inc. of Chattanooga, Tenn. (www.espintechnologies.com/company.htm) and others.

Single walled CNT have three different configurations that depend on the arrangement of the carbon hexagons around the tube circumference. When used in the creation of composite material, the dispersion and orientation of the SWCNT appear to be important factors in the overall structural properties of the formed material. The characteristics of varied carbon nanotube composites have been studied with the nanotube fraction ranging from less than 1% to about 25% of the total composite. The extensive and varied experimental results are listed in the previously cited references. In general, it appears that the physical characteristics of the composite are enhanced to a greater extent with the addition of MWCNT than with SWCNT. This is largely secondary to the increased stiffness of the MWCNT and the relative weakness of the van der Waals forces between individual SWCNT. The interaction between single walled CNT can be further enhanced by the addition of inter-tube bridging and this method is well described by A. Kis, et. al. in the article “Reinforcements of single-walled carbon nanotubes by inter-tube bridging” and published in Nature materials, Vol. 3, March, 2004. The text is hereby incorporated by reference in its entirety. Other methods of altering the physical performance of SWCNT composites include tube bonding to the matrix, formation of carbon “onions”, manipulation of the aspect ratio, irradiation and the like. The mechanical properties of carbon nanotubes are discussed further in the article “Mechanics of carbon nanotubes” by Dong Qian, et al and published in Appl. Mech. Rev. vol. 55, November, 2002. The text is hereby incorporated by reference in its entirety.

Because of their deformable nature, single walled CNT have been found to enhance the mechanical properties of elastomers. (See “Mechanical properties of carbon nanoparticle-reinforced elastomers” published in 2003 in Composite Science and Technology, 63, p1647. The text is hereby incorporated by reference in its entirety.) SWCNT appear to enhance the fairly modest wear characteristics of elastomers. This feature is particularly advantageous in the formulation of orthopedic implants that are adapted to oppose motion and bring the skeletal segment back to a neutral position after a motive force acting upon the skeleton has dissipated. These devices include dynamic implants of the spine and tendon replacement or augmentation implants. As an example, U.S. Pub. No. 2007/0027542 illustrates the design of an artificial ligament. The incorporation of nanotube technology would improve the mechanical properties, allow for use of a smaller prosthesis (for minimally invasive surgery) and provide for the introduction of reinforced elastomers that would enhance the resistive power while decreasing the device's antigenic qualities.

SWCNT appear to be excellent electrical conductors. Indeed, it now appears that formulation of nano-circuits may emerge as a significant use for carbon nanotubes. The electrical conductivity may be manipulated to fulfill particular functions with certain molecular modification. For example, recent studies have shown that titanium and other metals can be deposited on the outer walls of SWCNT. As the deposited titanium covers the outer tube surface, a stable conductive wire on the nano-scale is formed. (See “Binding energies and electronic structures of absorbed titanium chains on carbon nanotubes” by Yang et. al and published in Physical Review 66, 2002. The text is hereby incorporated by reference in its entirety.) The nanotubes function as templates for nanowire production and the attached metals alter the nanotube's physical properties thereby providing a powerful technique for the manipulation and refinement of electron transport by the wire.

Other methods of nano-conductor have been developed wherein carbon nanotubes were reacted with volatile oxide TiO at about 1375 C. The reaction produced a solid tubular nano-structure (“nano-rod”) that contained titanium and exhibited super-conducting properties. Further, Prabhakar Bandaru and colleagues at the University of California, San Diego produced Y-shaped MWCNT by chemical vapor deposition. The introduction of titanium-containing gases caused the nanotubes to branch and placed a catalytic particle at the branch junction. After placing platinum lead wires between the nanotubes and gold contact pads on a silicon substrate, they found that they could control the movement of electrons through the Y-junction by applying a voltage at its stem. This essentially creates an electrical switch or relay. The formation of a nano-scale switch and wires provides the building blocks for the development of electronic circuits. Indeed, Tesng et al have already reported the first nanotube-based integrated memory circuit. (Tesng et al “Monolithic Integration of Carbon Nanotube Devices with Silicon MOS Technology”, Nano Letters 4.1 (2004):123. The text is hereby incorporated by reference in its entirety.)

The conductive and superconductive properties of the nanotubes can be used in the formulation of NEMS within orthopedic implants. These systems can be used to measure, collect and transmit critical data about the neighboring tissues as well as to actively actuate the implanted device so as to produce a desired change within the skeletal system. These systems can be also used to generate and apply a current onto neighboring tissues in order to enhance healing. This is particularly applicable in tissues such as bone, where the application of an electrical current has been unequivocally shown to accelerate bone formation and healing.

Carbon nanotubes are capable of imparting electrical conductively to plastics and other materials. When compared to carbon fiber, stainless steel fibers and other additives, they impart an enhanced electrical conductivity to plastics. This important property can be easily exploited to manufacture smaller components, electrical and electronic components out of previously minimally conductive materials and to vary the magnetic properties of the implant. The last features can be used to manipulate the magnetic susceptibility of the device components and render them compatible with MRI imaging. (See Carbon nanotubes: A high performance conductive additive by Patrick Collins and John Hagerstrom at www.fibrils.com/PDFs/Perf%20Composites%20paper%202002-04-11.pdf. The text is hereby incorporated by reference in its entirety.)

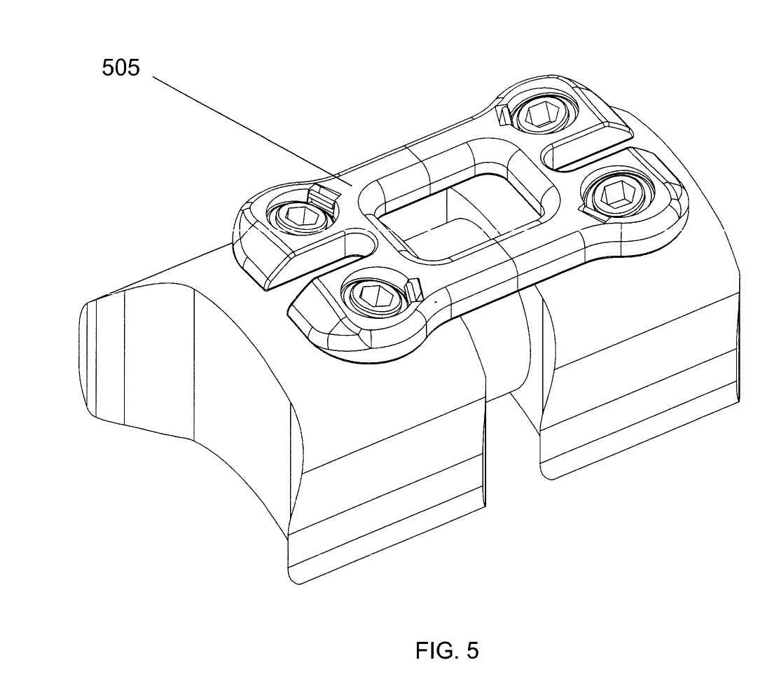

FIG. 5 illustrates the attachment of a bone-support device comprised of a plate 505 that uses bone screws or similar fasteners to attach onto the anterior aspect of the spine in cantilever fashion. The plate 505 includes one or more boreholes that accept bone screws for attaching the plate to first and second vertebrae. The plate 505 can be a least partially manufactured of nanotubes. Similar bone-support devices are widely used throughout the skeletal system. Depending on the skeletal region to be implanted, there may be significant limitation on the device size, shape or thickness. The skeletal system is surrounded by nerve elements, blood vessels and critical internal organs and the implantation of a bone-support device of reduced size is always advantageous. However, the device must be of sufficient strength in order to fulfill its role as a bone-supporting implant. In view of the preceding discussion, the use of nanotube-based composites in manufacture of the device enhances strength and allows a reciprocal reduction in device size. In addition, it may also provide an added advantage by permitting the formulation of a radio-lucent device.

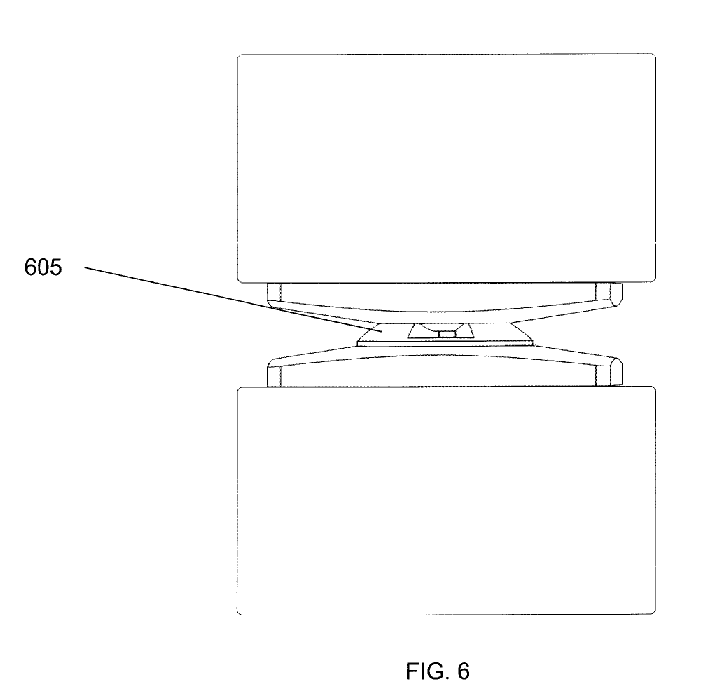

FIG. 6 illustrates a mobile prosthesis 605 that is situated between two skeletal segments and used to at least partially replace a joint structure. In the illustrated embodiment, the prosthesis is positioned in the disc space between two spinal segments. While shown within the disc space of a spinal segment, similar joint replacement devices are widely used in other skeletal joints. Critical factors in the longevity of joint replacement prosthesis' are the amount of wear debris produced by the bearing surfaces, and the inevitable device loosening and bone re-absorption that occur at the bone-device interface. The latter is at least partially caused by the former, since it's been unequivocally shown that the particulate debris from the bearing surfaces promote bone re-absorption and significantly accelerates device loosening. Once again, the preceding discussion has illustrated that the superior mechanical properties of nanotube-enhanced composites can significantly reduce the shedding of particulate debris from the composite bearing surface. Further, a nanotube-based NEMS sub-assembly can be incorporated within the joint replacement prosthesis to continuously measure the extent of bone re-absorption (so as to avoid sudden bone fracture and device failure), to actuate the device so as to redistribute the force more evenly across the re-absorbing bone and/or to apply a current to the reabsorbing bone so as to promote bone healing and reverse the re-absorptive process.

Since nanotubes have a hollow center, it is of interest to contemplate placement of foreign molecules or substances within that space. If successful, the housed substance can be used to impart new mechanical, electrical or magnetic properties onto the nanotube molecules. Further, the nanotube can be also used to transport and deliver the housed substance to a distant site and to simultaneously protect it during the transportation process. Filling nanotubes may be accomplished by several methods. The most useful method is to open the tube (at the caps or at the body) by chemical means and insert a foreign substance within the tube. Filled nanotubes may be particularly useful in orthopedic implants. They can be used to slowly deliver biologic factor such as Bone Morphogenic Protein (BMP) that expedite bone healing or other factors that modulate tissue healing. Alternatively, the device itself can be manufactured as a composite with filled nanotubes and the biologic factor contained within could promote device anchor and attachment onto the neighboring tissues.

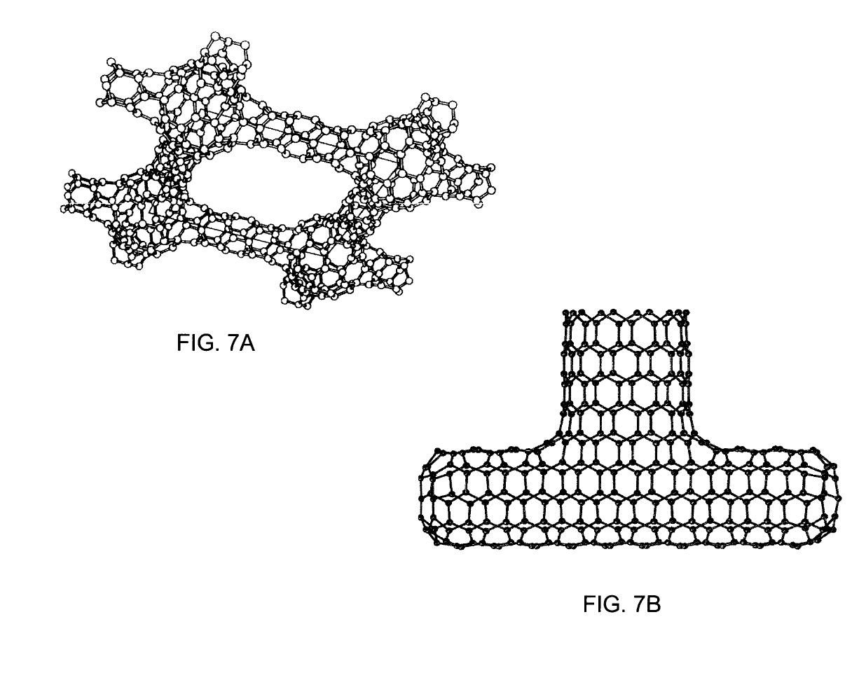

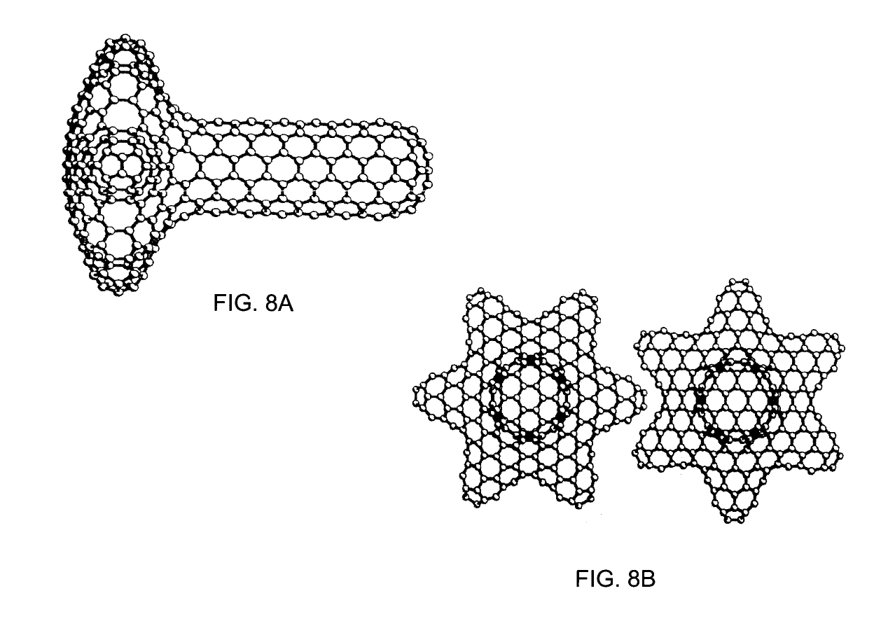

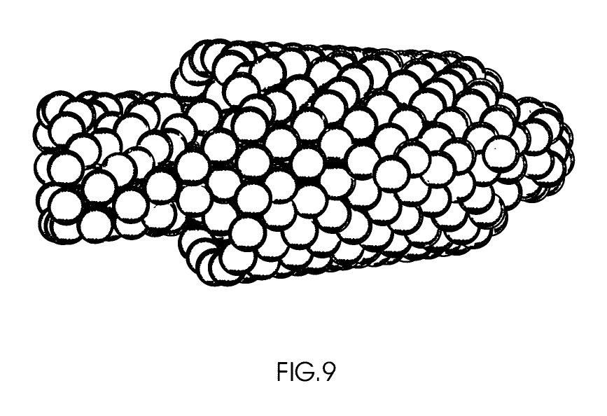

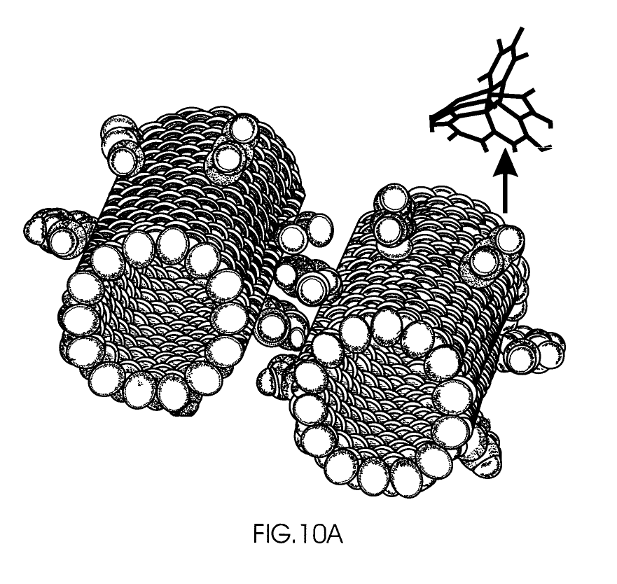

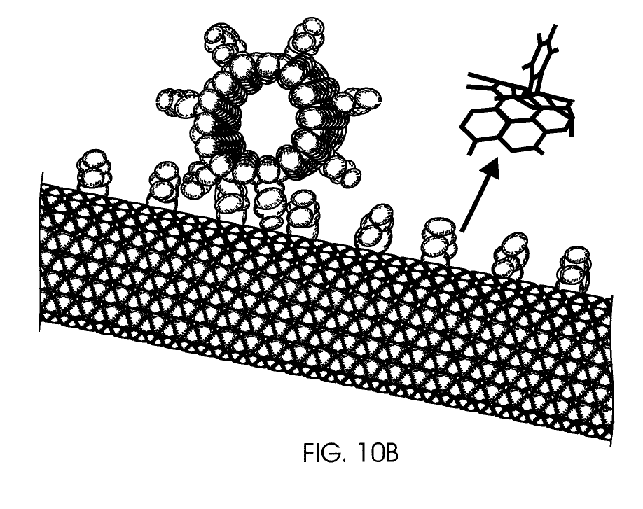

Future developments will undoubtedly bring additional shapes, the incorporation of additional non-carbon molecules and the production of devices on the nano-scale. FIGS. 7A and 7B show two additional nanotube shapes. FIGS. 8A and 8B illustrate side and top views of gear-like nano-tube device. FIG. 9 illustrates a nano-scale bearing surface. It is comprised of inner carbon nanotube housed within an outer toroid structure that has a central opening of sufficient diameter to accept the inner nano-tube. Finally, FIGS. 10A and 10B show a nano-tube based gear assembly that contains complimentary benzyne teeth. These and other potential future embodiments may be found in the aforementioned text “Carbon Nanotubes and Related Structures” by Peter J. F. Harris.

Nanotube technology in general and carbon nanotubes in particular have generated considerable research interest. Consequently, there have numerous patent application and patents granted that address this subject matter. While U.S. Patent Publication Nos. 2005/0203604 and 2004/0111141 illustrate the use of nanotube technology in the formulation of electrodes for use in implantable pacemakers and the like, there are no applications that illustrate or even contemplate the use of these materials in orthopedic implants.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed is:

1. A device for the stabilization of adjacent bony and ligamentous elements, comprising:
a body adapted to be at least partially situated between adjacent bony elements of a patient, the body defining at least one cavity adapted to contain a bone graft for bone fusion, wherein the body contains a superstructure adapted to support the load between the bony elements and wherein the body is at least partially manufactured from a nanotube.

2. A device as in claim 1, wherein the nanotube is a carbon nanotube.

3. A device as in claim 1, wherein the nanotube includes an inner cavity that contains additional elements of material.

4. A device as in claim 1, wherein the nanotube includes an outer structure and wherein additional elements of material are attached to the outer structure of the nanotube.

5. A device as in claim 1, wherein the nanotube is at least partially “Y” shaped

6. A device as in claim 1, wherein the nanotube is adapted to conduct electricity.

7. A device as in claim 1, wherein the device is adapted for use in the spine

8. A device as in claim 1, wherein the device is adapted for use in a skeletal site other than the spine.

9. A device for the stabilization of adjacent bony and ligamentous elements, comprising:
a body adapted to at least partially rest against a sidewall of a bony elements and affix to a pair of bony elements in a cantilevered fashion wherein the body is at least partially manufactured from a nanotube.

10. A device as in claim 9, wherein the nanotube is a carbon nanotube.

11. A device as in claim 9, wherein the nanotube includes an inner cavity that contains additional elements of material.

12. A device as in claim 9, wherein the nanotube includes an outer structure and wherein additional elements of material are attached to the outer structure of the nanotube.

13. A device as in claim 9, wherein the nanotube is at least partially “Y” shaped

14. A device as in claim 9, wherein the nanotube is adapted to conduct electricity.

15. A device as in claim 9, wherein the device is adapted for use in the spine

16. A device as in claim 9, wherein the device is adapted for use in a skeletal site other than the spine.

17. A device for the stabilization of a adjacent bony and ligamentous elements, comprising:
a body adapted to be at least partially situated between two bones wherein the body forms a bearing surface between the two bones wherein the body is at least partially manufactured from a nanotube.

18. A device as in claim 17, wherein the nanotube is a carbon nanotube.

19. A device as in claim 17, wherein the nanotube includes an inner cavity that contains additional elements of material.

20. A device as in claim 17, wherein the nanotube includes an outer structure and wherein additional elements of material are attached to the outer structure of the nanotube.

21. A device as in claim 17, wherein the nanotube is at least partially “Y” shaped

22. A device as in claim 17, wherein the nanotube is adapted to conduct electricity.

23. A device as in claim 17, wherein the device is adapted for use in the spine

24. A device as in claim 17, wherein the device is adapted for use in a skeletal site other than the spine.

25. A device for the stabilization of a adjacent bony and ligamentous elements, comprising:
a malleable tether used for stabilization of adjacent bones, wherein the tether provides the function of a natural ligament and wherein the tether is at least partially manufactured of a nanotube.

26. A device as in claim 25, wherein the nanotube is a carbon nanotube.

27. A device as in claim 25, wherein the nanotube includes an inner cavity that contains additional elements of material.

28. A device as in claim 25, wherein the nanotube includes an outer structure and wherein additional elements of material are attached to the outer structure of the nanotube.

29. A device as in claim 25, wherein the nanotube is at least partially “Y” shaped

30. A device as in claim 25, wherein the nanotube is adapted to conduct electricity.

31. A device as in claim 25, wherein the device is adapted for use in the spine

32. A device as in claim 25, wherein the device is adapted for use in a skeletal site other than the spine.
